(19) United States
(12) Patent Application Publication
(10) Pub. No.: US 2004/0236399 A1
(43) Pub. Date: Nov. 25, 2004

(54) Stent with improved surface adhesion

(75) Inventor: Rangarajan Sundar, Santa Rosa, CA (US)

Correspondence Address: FRANK C. NICHOLAS, CARDINAL LAW GROUP, Suite 2000, 1603 Orrington Avenue, Evanston, IL, 60201, US

(73) Assignee: Medtronic Vascular, Inc., Santa Rosa, CA

(21) Appl. No.: 10/829,511

(22) Filed: Apr. 22, 2004

Related U.S. Application Data
(60) Provisional application No. 60/464,440, filed on Apr. 22, 2003.

(51) Int. Cl. A61F 2/06

(52) U.S. Cl. 623/1.11; 623/1.46; 606/194; 623/901

(57) ABSTRACT

The stent with improved surface adhesion of the present invention increases adhesion between a stent and a coating by including a silane layer between the stent and the coating. The silane layer improves the wettability of the stent for application of a liquid coating and increases the coating adhesion. A coated stent can be manufactured by applying a silane solution to a stent by spraying or dipping the stent to form a silane layer on the stent surface, curing the resulting silane layer in an inert atmosphere, and applying a coating to the silane layer. In one embodiment, an amino silane can be used with a stainless steel stent.

### RELATED APPLICATION

This application claims priority to U.S. Provisional Application No. 60/464,440, “Stent with Improved Surface Adhesion” to Rangarajan Sundar, filed Apr. 22, 2003, the entirety of which is incorporated by reference.

### TECHNICAL FIELD

The technical field of this disclosure is medical implant devices, particularly, a stent having improved surface adhesion of coatings.

### BACKGROUND OF THE INVENTION

Stents are generally cylindrical shaped devices that are radially expandable to hold open a segment of a blood vessel or other anatomical lumen after implantation into the body lumen. Stents have been developed with coatings to deliver drugs or other therapeutic agents.

Stents are used in conjunction with balloon catheters in a variety of medical therapeutic applications including intravascular angioplasty. For example, a balloon catheter device is inflated during PTCA (percutaneous transluminal coronary angioplasty) to dilate a stenotic blood vessel. The stenosis may be the result of a lesion such as a plaque or thrombus. After inflation, the pressurized balloon exerts a compressive force on the lesion thereby increasing the inner diameter of the affected vessel. The increased interior vessel diameter facilitates improved blood flow. Soon after the procedure, however, a significant proportion of treated vessels re-narrow.

To prevent restenosis, short flexible cylinders, or stents, constructed of metal or various polymers are implanted within the vessel to maintain lumen size. The stents acts as a scaffold to support the lumen in an open position. Various configurations of stents include a cylindrical tube defined by a mesh, interconnected stents or like segments. Some exemplary stents are disclosed in U.S. Pat. No. 5,292,331 to Boneau, U.S. Pat. No. 6,090,127 to Globerman, U.S. Pat. No. 5,133,732 to Wiktor, U.S. Pat. No. 4,739,762 to Palmaz and U.S. Pat. No. 5,421,955 to Lau. Balloon-expandable stents are mounted on a collapsed balloon at a diameter smaller than when the stents are deployed. Stents can also be self-expanding, growing to a final diameter when deployed without mechanical assistance from a balloon or like device.

Stents have been used with coatings to deliver drug or other therapy at the site of the stent. The coating can be applied as a liquid containing the drug or other therapeutic agent dispersed in a polymer/solvent matrix. The liquid coating then dries to a solid coating upon the stent. The liquid coating can be applied by dipping or spraying the stent while spinning or shaking the stent to achieve a uniform coating. Combinations of the various application techniques can also be used.

Problems arise in getting coatings to adhere to stents, particularly stents made of stainless steel. Polymers such as urethanes, polyesters, epoxies, and the like do not easily wet stainless steel, so it is difficult to achieve contact between the coating and the stent surface. The incorporation of drugs in the polymers further reduces wettability. Wettability can be improved by methods such as exposing the stent surface to electrical corona or plasma, but the surface energy from such methods dissipates quickly, limiting the time when the stent can be coated. Plasma and corona treatment are most beneficial for polymeric materials, where the surfaces can be modified to become chemically reactive, but are ineffectual for bare metal surfaces. This limits the usefulness of such methods for large scale manufacturing operations.

Wettability can also be improved by the use of various primer coatings, such as acrylates, urethanes, phosphates, silicones, and polyesters, but these primer coatings need to be very thin and must be compatible with the drug-bearing coating or other intermediate coating applied on top of it. Another problem with primer coatings is that such coatings are prone to pooling and bridging, i.e., prone to filling in spaces in the stent that should be open and to forming thick spots in the coating. The rheology of primer coatings must be precisely controlled to obtain a clean surface for adhesion of the outer coating.

It would be desirable to have a stent having stent having improved surface adhesion that would overcome the above disadvantages.

### SUMMARY OF THE INVENTION

One aspect of the present invention provides a stent having improved surface adhesion to increase coating adherence.

Another aspect of the present invention provides a stent having improved surface adhesion, avoiding the need for a primer coat.

Another aspect of the present invention provides a stent having improved surface adhesion in which wettability is maintained for a sufficient time to allow coating application.

Another aspect of the present invention provides a stent having improved surface adhesion to improve ease of manufacture.

The foregoing and other features and advantages of the invention will become further apparent from the following detailed description of the presently preferred embodiments, read in conjunction with the accompanying drawings. The detailed description and drawings are merely illustrative of the invention, rather than limiting the scope of the invention being defined by the appended claims and equivalents thereof.

### BRIEF DESCRIPTION OF THE DRAWINGS

FIG. 1 shows a stent delivery system made in accordance with the present invention;

FIG. 2 shows a stent made in accordance with the present invention; and

FIG. 3 shows a method of manufacturing a stent made in accordance with the present invention.

### DETAILED DESCRIPTION OF THE PRESENTLY PREFERRED EMBODIMENTS

The stent with improved surface adhesion of the present invention increases adhesion between a stent and a coating by including a silane layer between the stent and the coating. The silane layer improves the wettability of the stent for application of a liquid coating and increases coating adhesion. A coated stent can be manufactured by applying a silane solution to a stent by spraying or dipping the stent to form a silane layer on the stent surface, curing the resulting silane layer in an inert atmosphere, and applying a coating to the silane layer. In one embodiment, an amino silane can be used with a stainless steel stent.

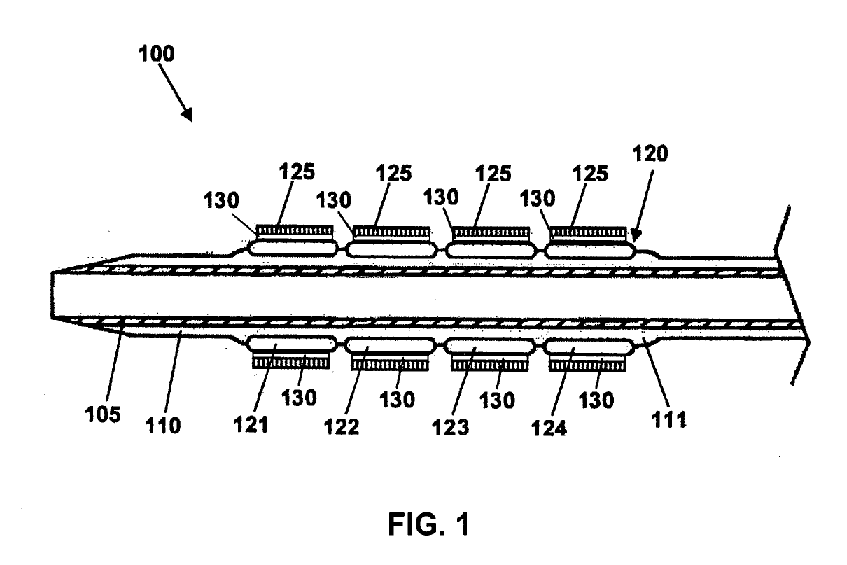

FIG. 1 shows a stent delivery system made in accordance with the present invention. The stent delivery system 100 includes a catheter 105, a balloon 110 operably attached to the catheter 105, and a stent 120 disposed on the balloon 110. The balloon 110, shown in a collapsed state, may be any variety of balloons capable of expanding the stent 120. The balloon 110 may be manufactured from any suitable material such as polyethylene, polyethylene terephthalate (PET), nylon, or the like. In one embodiment, the balloon 110 may include retention means 111, such as mechanical or adhesive structures, for retaining the stent 120 until it is deployed. The catheter 105 may be any variety of balloon catheters, such as a PTCA (percutaneous transluminal coronary angioplasty) balloon catheter, capable of supporting a balloon during angioplasty.

The stent 120 may be any variety of implantable prosthetic devices capable of carrying a coating known in the art. In one embodiment, the stent 120 may have a plurality of identical cylindrical stent segments placed end to end. Four stent segments 121, 122, 123, and 124 are shown, and it will be recognized by those skilled in the art that an alternate number of stent segments may be used. The stent 120 includes at least one coating 125 carrying a therapeutic agent, which can be applied to the stent 120 by dipping or spraying the stent 120 with a coating liquid, or applying the coating liquid with a combination of methods. The coating can be applied as a liquid containing the drug or other therapeutic agent dispersed in a polymer/solvent matrix. In another embodiment, the therapeutic agent can be omitted from the coating and the coating included for its mechanical properties.

A thin silane layer 130 between the coating 125 and the stent 120 acts as a connection to promote adhesion between the organic coating 125 and the inorganic metal stent 120. The coating 125 is merely exemplary, and it should be recognized that other coating configurations, such as multiple coating layers, are possible. Although the coating 125 and the silane layer 130 are shown schematically on the outer circumference of the stent 120, the coating 125 and the silane layer 130 can coat the whole stent 120, both inside and outside, and around the cross section of individual stent segment wires. In another embodiment, the silane layer 130 can be present on a portion of the stent 120 without a coating 125 on that same portion.

The coating 125 can be a polymer including, but not limited to, urethane, polyester, epoxy, polycaprolactone (PCL), polymethylmethacrylate (PMMA), PEVA, PBMA, PHEMA, PEVAc, PVAc, Poly N-Vinyl pyrrolidone, Poly (ethylene-vinyl alcohol), combinations of the above, and the like. Suitable solvents that can be used to form the liquid coating include, but are not limited to, acetone, ethyl acetate, tetrahydrofuran (THF), chloroform, N-methylpyrrolidone (NMP), combinations of the above, and the like. Suitable therapeutic agents include, but are not limited to, antiangiogenesis agents, antiendothelin agents, antimitogenic factors, antioxidants, antiplatelet agents, antiproliferative agents, antisense oligonucleotides, antithrombogenic agents, calcium channel blockers, clot dissolving enzymes, growth factors, growth factor inhibitors, nitrates, nitric oxide releasing agents, vasodilators, virus-mediated gene transfer agents, agents having a desirable therapeutic application, combinations of the above, and the like. Specific example of therapeutic agents include abciximab, angiopeptin, colchicine, eptifibatide, heparin, hirudin, lovastatin, methotrexate, streptokinase, taxol, ticlopidine, tissue plasminogen activator, trapidil, urokinase, and growth factors VEGF, TGF-beta, IGF, PDGF, and FGF.

In a further embodiment, the drug coating composition may be fashioned using the drug 42-Epi-(tetrazolyl)-rapamycin, set forth in U.S. Pat. No. 6,329,386 assigned to Abbott Laboratories, Abbott Park, Ill., and dispersed within a coating fashioned from phosphorylcholine coating of Biocompatibles International plc, set forth in U.S. Pat. No. 5,648,442. Other emobidments may be fashioned from rapamycin analogs or rapamycin derivatives.

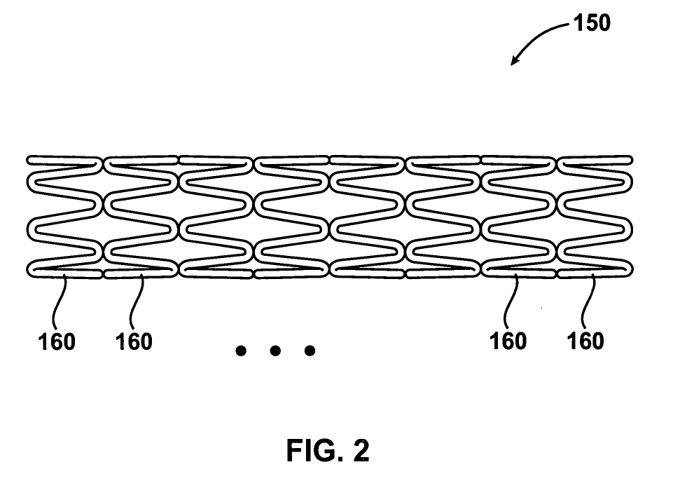

FIG. 2 shows a stent made in accordance with the present invention. The stent 150 comprises a number of segments 160. The pattern of the segments 160 can be W-shaped or can be a more complex shape with the elements of one segment continuing into the adjacent segment. The stent 150 can be installed in the stent delivery system of FIG. 1 for implantation in a body lumen.

Referring to FIG. 2, the stent 150 is conventional to stents generally and can be made of a wide variety of medical implantable materials, such as stainless steel (particularly 316-L stainless steel or 316LS), MP35 alloy, nitinol, tantalum, ceramic, nickel, titanium, aluminum, polymeric materials, tantalum, MP35N, titanium ASTM F63-83 Grade 1, niobium, high carat gold K 19-22, and combinations thereof. The stent 150 can be formed through various methods as well. The stent 150 can be welded, laser cut, molded, or consist of filaments or fibers which are wound or braided together in order to form a continuous structure. Depending on the material, the stent can be self-expanding, or be expanded by a balloon or some other device. The silane layer and coating can be on the surface of the segments 160.

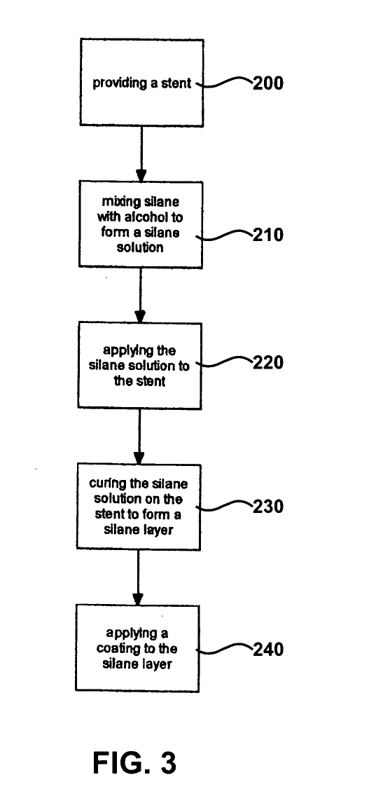

FIG. 3 shows a method of manufacturing a stent made in accordance with the present invention. At 200, a stent is provided. Silane is mixed with alcohol 210 and applied to the stent 220. The stent is cured to form a silane layer 230 and a coating applied over the silane layer 240.

To establish an amino silane layer on a stent, a solution of amino silane in a solvent is applied to the stent, the solution is held in contact with the stent for a selected time at a selected temperature, and the amino silane layer is cured in an inert atmosphere for a selected time at a selected temperature. An alcohol wash can be used to remove excess silane before curing. The coating topography of the amino silane layer and the adhesion properties can be controlled by varying factors such as stent surface preparation, solution contact time, amino silane solution concentration, and curing temperature.

The amino silane layer functions as a connection between an inorganic metal substrate of the stent and the organic polymer coating. The thin layer of amino silane is covalently bonded to the stent and functions as an effective adhesion promoter. The amino silane layer can be a monolayer, multilayer, or bulk phase of amino silane on the stainless steel stent. Typically, the silane layer thickness will be extremely thin, on the order of 8-10 monolayers.

Different silanes can be used with stents, particularly amino silanes, and more particularly trimethoxysilylpropyl-diethylenetriamine; 3 aminopropyltrimethoxysilane; n-styrylmethyl 2 aminoethylamino propyl trimethoxysilane; vinyl trimethoxysilane; methacryloxypropyltrimethoxysilane; 3-(n-styrylmethyl-2-aminoethylaminopropyltrimethoxysilane); or 3 (glycidoxypropyl)-trimethoxysilane. The characteristic that allows the silanes to form a connection layer is the ability to covalently link with the stent surface. In general, silanes are chosen that will have a good affinity with both metal and organic surfaces.

The general formula of an organosilane is RₙSiX₍₄₋ₙ₎. The X group is involved in the reaction with the inorganic substrate, in this case, the stent surface. The bond between X and the silicon atom in the silane solution is replaced by a bond between the inorganic substrate and the silicon atom. X is a hydrolyzable group, such as alkoxy, acyloxy, amine, or chlorine. The most common alkoxy groups are methoxy and ethoxy. R is a nonhydrolyzable organic radical that possesses a functionality which enables the silane layer to bond with organic resins and polymers.

Reaction of silanes to form a silane layer involves four steps. Initially, hydrolysis of the three labile X groups attached to silicon occurs. Condensation to oligomers follows. The oligomers then hydrogen bond with OH groups of the substrate. Finally, during drying or curing, a covalent linkage is formed with the substrate with concomitant loss of water.

The solvent in which the amino silane is mixed prior to application to the stent can be an alcohol, such as isopropyl alcohol, methyl alcohol, ethyl alcohol, or the like. The amount of amino silane added to the solution can be from 2-30%. Amino silane concentration in the solution affects the quality and quantity of amino silane deposition on the stent surface. A very high concentration of 10-15% gives a rougher topography and a thicker silane layer whereas a lower concentration of 2-5% gives a smoother topography and a thinner silane layer. The differences in smoothness can be seen by scanning electron microscope. A compromise between thickness and smoothness of the amino silane layer is necessary to obtain good adhesion of the coating. While the thicker amino silane layer provides greater coating holding power, the surface roughness carries through to the outer coating surface. Outer coating surface roughness may be undesirable in certain applications depending on the coating interaction with the body lumen in which it is placed and the fluids in the body lumen.

The silane solution must be properly prepared prior to application. After the silane is mixed with the alcohol, the silane solution must be allowed time to hydrolyze and stabilize before application to the stent. Water for hydrolysis can be added to the silane solution or can come from the atmosphere. Typically, a time of about 10 to 30 minutes is sufficient for hydrolyzation and stabilization, particularly a time of about 15 minutes. For some silanes, such as vinyltrimethoxysilane for example, the pH of the silane solution must be adjusted to the 4-5 range by the addition of acids, such as acetic acid, for the solution to be effective.

The variation of silane layer thickness with silane concentration in the silane solution has been confirmed experimentally. Since the silane is deposited as a very thin layer, sophisticated analytical techniques such as Time of Flight Secondary Ion Mass Spectroscopy (TOF-SIMS) or X-Ray Photoelectron Spectroscopy (XPS) are required to determine the silane layer thickness. In one test, a steel coupon of 316L stainless steel was immersed in a low concentration amino silane solution (2%) and another steel coupon of 316L stainless steel was immersed high concentration amino silane solution (10%). The samples were washed and cured in an inert atmosphere for 24 hours. Using TOF-SIMS to test for the presence of amino silane, the lower concentration sample showed low signal content signifying a low silane layer thickness, while the higher concentration sample showed a very high signal indicating a much thicker layer.

The silane solution can be applied to the stent by various methods, such as dip coating or spraying, brushing, or wiping. The stent surface can be prepared to be free from dirt or oil, but some oxidation on the surface is desirable to provide points of adhesion for the silane layer. Surface pretreatment with detergents and ultrasonic cleaning, or the like can also improve the silane layer adhesion. Dip coating has the advantage of providing a more uniform silane layer. For dip coating, the time of immersion in the silane solution affects silane layer thickness and topography. A short immersion time of 5 minutes results in a thin silane layer with a smooth surface, while a long immersion time such as 60 minutes results in a thick silane layer with a rough surface. Typically, an immersion time between about 1 to 60 minutes is desirable, particularly a time of about 15 minutes. An immersion bath temperature of about 20 to 70 deg.C is desirable, particularly an immersion bath temperature of about 35 deg.C. Excess silane can be removed from the stent by washing the stent in alcohol and water.

Curing time and temperature affect the covalent bond formation between the silane layer and the stent surface. An inert cover gas, such as argon or nitrogen, can be used to prevent oxidation and provide an effective cure. Curing time and temperature vary inversely: a lower curing temperature requires a longer curing time. Experiments have shown that a curing time of 24 hours is needed for a curing temperature of 77 degrees F., while a curing time of 15 minutes is sufficient for a curing temperature of 100 degrees F. Typically, a curing time of about 1 to 24 hours with a curing temperature of about 25 to 115 deg.C is desirable, particularly a curing time of about 3 to 15 hours with a curing temperature of about 60 deg.C. Those skilled in the art will appreciate that the curing time and temperature need to be optimized for the materials involved, the results desired, and manufacturing constraints. The treated stents can be used up to 3 to 6 months after treatment if stored in a suitable environment to prevent surface contamination.

Experimental results show that the silane layer improves the wettability of stent material. Coupons made from the same 316L, 316LS, or MP35 stock from which stents are manufactured were used to make contact angle measurements. Contact angle measurements are typically carried out by the Sessile drop method or by the use of a Dynamic Contact Angle Analyzer. With no treatment, the contact angle was high at about 60-80 degrees. By treating coupons with amino silane, the contact angle was lowered to 15-20 degrees, showing the increased wettability from application of the silane layer.

Experimental results also show that the silane layer improves adhesion of the polymer coating. An ASTM D-3359 cross hatch adhesion test was used to determine the effectiveness of the amino silane layer. In the test, the polymer coating is scored in a cross-hatched pattern forming squares, an adhesive tape applied to the polymer coating surface, the adhesive tape pulled away, and the fraction of squares suffering lost or damaged coating determined. Experimental samples treated with amino silane suffered a negligible loss of adhesion, whereas samples without amino silane treatment showed a significant amount of adhesion failure between the polymer coating and the stainless steel.

It is important to note that FIGS. 1-3 illustrate specific applications and embodiments of the present invention, and is not intended to limit the scope of the present disclosure or claims to that which is presented therein. For example, many combinations of silanes, alcohols, concentrations, times, and temperatures can be used to achieve a silane layer increasing adhesion between the polymer coating and the stent material. Upon reading the specification and reviewing the drawings hereof, it will become immediately obvious to those skilled in the art that myriad other embodiments of the present invention are possible, and that such embodiments are contemplated and fall within the scope of the presently claimed invention.

While the embodiments of the invention disclosed herein are presently considered to be preferred, various changes and modifications can be made without departing from the spirit and scope of the invention. The scope of the invention is indicated in the appended claims, and all changes that come within the meaning and range of equivalents are intended to be embraced therein.

1. A stent delivery system comprising:
a catheter;
a balloon operably attached to the catheter; and
a stent disposed on the balloon;
a silane layer disposed on the stent; and
a coating disposed on the silane layer.

2. The stent delivery system of claim 1 wherein the coating includes a therapeutic agent.

3. The stent delivery system of claim 1 wherein the coating is a polymer.

4. The stent delivery system of claim 1 wherein the silane layer is selected from the group consisting of a monolayer, a multilayer, and a bulk phase layer.

5. The stent delivery system of claim 1 wherein the stent is a stainless steel stent.

6. A coated stent comprising:
a stent;
a silane layer disposed on the stent; and
a coating disposed on the silane layer.

7. The coated stent of claim 6 wherein the coating includes a therapeutic agent.

8. The coated stent of claim 6 wherein the coating is a polymer.

9. The coated stent of claim 6 wherein the silane layer is selected from the group consisting of a monolayer, a multilayer, and a bulk phase layer.

10. The coated stent of claim 6 wherein the stent is a stainless steel stent.

11. A method for producing a stent comprising:
providing a stent;
mixing silane with alcohol to form a silane solution;
applying the silane solution to the stent; and
curing the silane solution on the stent to form a silane layer.

12. The method of claim 11 further comprising applying a coating to the silane layer.

13. The method of claim 12 wherein the coating includes a therapeutic agent.

14. The method of claim 12 wherein the coating is a polymer.

15. The method of claim 11 wherein the stent is a stainless steel stent.

16. The method of claim 11 wherein the silane is an amino silane.

17. The method of claim 11 wherein the silane is selected from the group consisting of trimethoxysilylpropyl-diethylenetriamine; 3 aminopropyltrimethoxysilane; n-styrylmethyl 2 aminoethylamino propyl trimethoxysilane; vinyl trimethoxysilane; methacryloxypropyltrimethoxysilane; 3-(n-styrylmethyl-2-aminoethylaminopropyltrimethoxysilane); and 3 (glycidoxypropyl)-trimethoxysilane.

18. The method of claim 11 wherein the alcohol is selected from the group consisting of isopropyl alcohol, methyl alcohol, and ethyl alcohol.

19. The method of claim 11 wherein mixing silane with alcohol to form a silane solution further comprises mixing silane with alcohol to form a silane solution of about 2 to 30% silane.

20. The method of claim 11 wherein mixing silane with alcohol to form a silane solution further comprises mixing silane with alcohol to form a silane solution of about 5% silane.

21. The method of claim 11 wherein applying the silane solution to the stent further comprises dipping the stent in the silane solution.

22. The method of claim 11 wherein applying the silane solution to the stent further comprises spraying the stent with the silane solution.

23. The method of claim 11 wherein applying the silane solution to the stent further comprises applying the silane solution at a temperature between about 20 to 70 deg.C for a time of between about 1 and 60 minutes.

24. The method of claim 11 wherein applying the silane solution to the stent further comprises applying the silane solution at a temperature of about 35 deg.C for a time of about 15 minutes.

25. The method of claim 11 wherein curing the silane solution on the stent to form a silane layer further comprises curing the silane solution in an inert atmosphere.

26. The method of claim 11 wherein curing the silane solution on the stent to form a silane layer further comprises curing the silane solution at a temperature between about 25 to 115 degrees C. for a time of between about 1 to 24 hours.

27. The method of claim 11 wherein curing the silane solution on the stent to form a silane layer further comprises curing the silane solution at a temperature of about 60 degrees C. for a time of about 3 to 15 hours.

28. A system for producing a stent comprising:
means for providing a stent;
means for mixing silane with alcohol to form a silane solution;
means for applying the silane solution to the stent; and
means for curing the silane solution on the stent to form a silane layer.

29. The system of claim 28 further comprising means for applying a coating to the silane layer.

30. The system of claim 28 wherein means for applying the silane solution to the stent further comprises means for dipping the stent in the silane solution.

31. The system of claim 28 wherein means for applying the silane solution to the stent further comprises means for spraying the stent with the silane solution.

32. The system of claim 28 wherein the means for curing the silane solution on the stent to form a silane layer further comprises means for curing the silane solution in an inert atmosphere.

33. A coated stent comprising:
a stainless steel stent;
an amino silane layer disposed on the stainless steel stent;
a polymer coating disposed on the amino silane layer, the polymer coating including a therapeutic agent.
